(19) United States
(12) Patent Application Publication
(10) Pub. No.: US 2007/0041917 A1
(43) Pub. Date: Feb. 22, 2007

(54) Sunless tanning compositions

(76) Inventor: Isaac Thomas, Carrollton, TX (US)

Correspondence Address: FULBRIGHT & JAWORSKI L.L.P., 600 CONGRESS AVE., SUITE 2400, AUSTIN, TX, 78701, US

(21) Appl. No.: 11/474,139

(22) Filed: Jun. 23, 2006

Related U.S. Application Data
(60) Provisional application No. 60/708,888, filed on Aug. 17, 2005.

(51) Int. Cl. A61K 8/89

(52) U.S. Cl. 424/59; 424/70.12

(57) ABSTRACT

A sunless skin tanning emulsion is disclosed. The emulsion can be a water-in-silicon emulsion and can include a skin tanning agent and a silicon containing compound comprising the formula: 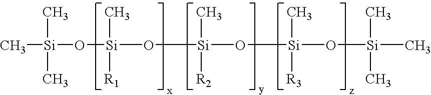
wherein, R₁, R₂, R₃, are independently CH₃, [CH₂]₃—[OCH₂CH₂]_{w}—OH, or [CH₂]ₘ-A. Also disclosed are methods of using the emulsions in sunless skin tanning applications.

### CROSS REFERENCE TO RELATED APPLICATIONS

This application claims the benefit of U.S. Provisional Application No. 60/708,888, filed Aug. 17, 2005, the contents of which are incorporated by reference.

### BACKGROUND OF THE INVENTION

A. Field of the Invention

The present invention relates generally to compositions and methods for their use in skin-tanning applications. In particular, the compositions can, for example, be formulated as water-in-silicone emulsions that include a sunless skin tanning agent and a silicone containing surfactant. These compositions can be used to tan or darken skin without the need for exposing the skin to sun or artificial ultraviolet (UV) light sources such as UV lamps.

B. Background of the Invention

Enjoying the sun has been a favorite pastime for people of all ages for many years. A product of enjoying the sun is obtaining a tan. Many societies associate darkened or tanned skin with health and beauty. Unfortunately, sun exposure can be damaging to the skin and has been shown to cause wrinkles, brown age spots, blotchiness, and leathery, sagging skin. In worst-case scenarios, over-exposure to the sun can cause skin cancer which can be disfiguring and even deadly.

Sunless tanning agents can be used to obtain a tan without the need to expose skin to the damaging rays of the sun. For instance, products containing dihydroxyacetone (“DHA”) have been marketed for several decades as being able to darken the skin without the need for sun exposure. DHA is a colorless sugar that interacts with dead skin cells in the stratum corneum of the epidermis. The skin cells change color because of this interaction thereby giving the appearance of a sun tan. The color change typically lasts for about 5 to 7 days and will gradually fade.

The cosmetic appeal of a sunless skin tanning composition is an important feature. Previous compositions can lack such an appeal because they have, for example, a sticky, oily, greasy, gritty, or an overall unpleasant feeling when applied to the skin. Additionally, many compositions are difficult to apply (e.g., do not rub into the skin easily) and do not disappear after skin application. Other compositions have an aesthetically un-pleasing visual appearance or fragrance. In some cases, sunless tanning compositions have been found to irritate the skin.

### SUMMARY OF THE INVENTION

The inventor has overcome the deficiencies in the art by providing compositions and methods for there use in sunless tanning applications. The compositions of the present invention have improved visual and tactile properties and can be easily applied to skin.

In one aspect of the present invention, for example, there is disclosed a water-in-silicon sunless skin tanning emulsion that includes a skin tanning agent and a silicone containing compound comprising the formula: 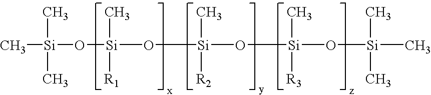
wherein, R₁, R₂, R₃, are independently CH₃, [CH₂]₃—[OCH₂CH₂]_{w}—OH, or [CH₂]ₘ-A, wherein; w is an integer between 2-20; and A is: 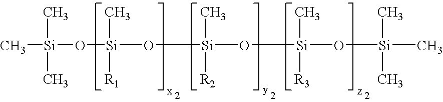
and wherein x, y, z, x₂, y₂, z₂, and m are independently an integer between 1 and 1000, 10 and 500, 20 and 100, or 30 and 50, or any integer derivable therein (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, and 999). In other aspects, the integer for x, y, z, x₂, y₂, z₂, and m can independently be more than 1000 (e.g., 1,500, 2,000, 3,000, 4,000, 5,000, or more).

In particular embodiments, the silicon containing compound is a dimethicone crosslinked polymer comprising the formula: 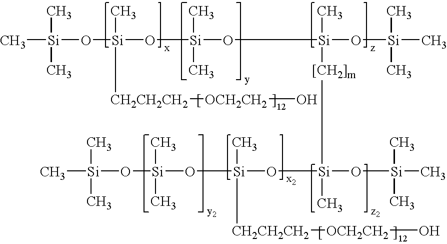
wherein x, y, z, x₂, y₂, z₂, and m are independently an integer between 1 and 1000, 10 and 500, 20 and 100, or 30 and 50, or any integer derivable therein (e.g., see the integers listed from 2-999 above). In other aspects, the integer for x, y, z, x₂, y₂, z₂, and m can independently be more than 1000 (e.g., 1,500, 2,000, 3,000, 4,000, 5,000, or more).

The emulsion can comprise, in other non-limiting embodiments, from about 2% to about 10% by weight of the silicon containing compound relative to the total weight of the emulsion. In other aspects, the emulsion comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more by weight of the silicon containing compound relative to the total weight of the emulsion.

The skin tanning agent, in preferred aspects, is DHA. It is contemplated that other skin tanning agents can be used in the place of or in combination with DHA. Non-limiting examples include bronzers, pigmentation agents (e.g., methoxsalen, trioxsalen, and melanin), dyes, botanical extracts (e.g., silver birch (Betulla alba), and Mahakanni STLC (Eclipta alba)), and chemical compounds (e.g. erythrulose, lawsone, tyrosine, orjugulone, alpha-hydroxy aldehydes and ketones, glyceraldehyde and related alcohol aldehydes, various indoles, imidazoles, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde). In certain aspects, the emulsion can include from about 5% to about 10% by weight of the skin tanning agent relative to the total weight of the emulsion. As noted throughout this documents, however, the amounts of the skin tanning agent in an emulsion of the present invention can vary. For example, the amount can include, by percentage, 1, 2, 3, 4, 5, 6, 7, 8, 9, 10, 15, 20, 25, 30, 35, 40, 45, 50, 60, 70, 80, 90, 95, or more, by weight or volume of the skin tanning agent relative to the total weight or volume of the emulsion.

In other non-limiting aspects, the emulsions of the present invention can be included in a cosmetic composition or product. The emulsions, compositions, or products of the present invention can be transparent, semi-transparent, clear, or non-transparent. The emulsions, compositions, or products can also be colored (e.g., pink, white, off-white, pearl, brown, tan, creme). In certain preferred aspects, the emulsion is formulated into a transparent or semi-transparent gel.

The emulsions can further include a cyclic or non-cyclic siloxane. In certain non-limiting aspects, for example, the cyclic siloxane can be cyclomethicone (CAS number 69430-24-6), cyclopentasiloxane (CAS number 6166-86-5), decamethylcyclopentasiloxane (CAS number 541-02-6), cyclohexasiloxane, cyclotetrasiloxane, or any other cyclic siloxanes that are known to those of skill in the art and are useful within the context of the present invention. Non-limiting examples of non-cyclic siloxanes include dimethicone, C₃₀₋₄₅ alkyl dimethicone, cetearyl methicone, cetyl dimethicone, and any other non-cyclic siloxanes that are known to those of skill in the art and are useful within the context of the present invention.

The emulsions of the present invention can include at least two cyclic or non-cyclic siloxanes, or a combination of cyclic and non-cyclic siloxanes. In preferred aspects, the emulsions include cyclomethicone and cyclopentasiloxane or cyclomethicone and decamethylcyclopentasiloxane. In certain aspects, the emulsion can include from about 5% to about 30% by weight of a cyclic or-non-cyclic siloxane relative to the total weight of the emulsion. In other aspects, the emulsion comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 60%, 70%, 80%, 90% or more by weight or volume of the cyclic or non-cyclic siloxane relative to the total weight or volume of the emulsion. The amounts of the cyclic or-non-cyclic siloxane in an emulsion of the present invention can vary.

The emulsions of the present invention, in other non-limiting aspects, can include a mono or dihydric alcohol. Non-limiting examples of monohydric alcohols that are contemplated as being used with the present invention include ethanol, isopropanol, and any other monohydric alcohols that are known to those of ordinary skill in the art and are useful within the context of the present invention. Non-limiting examples of dihydric alcohols that are contemplated as being used with the present invention include butylene glycol, propylene glycol, isopentyldiol, and any other dihydric alcohols that are known to those of ordinary skill in the art and are useful within the context of the present invention. In preferred aspects, the emulsion includes about 3% to about 12% by weight of the monohydric or dihydric alcohol relative to the total weight or volume of the emulsion. The amounts of the monohydric and/or di-hydric alcohol in an emulsion of the present invention can vary. For example, the amount can include, by percentage, 1, 2, 3, 4, 5, 6, 7, 8, 9, 10, 15, 20, 25, 30, 35, 40, 45, 50, 60, 70, 80, 90, 95, or more, by weight or volume of the monohydric and/or di-hydric alcohol relative to the total weight or volume of the emulsion.

In yet another non-limiting embodiment of the present invention, the water-in-silicon sunless skin tanning emulsion can include a gellant. Non limiting examples of gellants that can be used with the present invention include dibenzylidene alditols, carboxylated salts, polysaccharides, protein/polysaccharide complexes, or mixtures thereof.

In preferred embodiments, compositions of the present invention are formulated into water-in-silicone emulsions. It is contemplated, however, that other types of emulsions can be used. Non-limiting examples include silicone-in-water, water-in-oil, oil-in-water, water-in-silicone in water, and silicone-in-water in silicon emulsions. In other aspects, the inventor also contemplates the use of microemulsions including, for example, single phase (e.g., bicontinuous phase of water and oil or water and silicon), two phase (e.g., oil-in-water, water-in-oil, silicon-in-water, water-in-silicon), and three phase (e.g oil or silicon, water, and a middle bicontinuous microemulsion phase coexisting in a three-phase equilibrium). Microemulsions have the ability to allow the mixing of water and oil or silicon in a thermodynamically stable state without the use of mechanical agitation to produce the single-phase solution.

In other non-limiting aspects, the compositions of the present invention can include a color additive as described throughout this specification and incorporated into this section by reference. The color additive can be used to impart a particular color (e.g., white, off-white, brown (including light brown, dark brown, tan, crème, caramel, and copper), yellow, orange, blue, green, red, etc.) to the composition. In certain non-limiting embodiments, the color additive provides the composition with a brown tint. The brown tint can resemble the color of tanned skin. When the composition is applied to the skin (application can include but is not limited to spreading on and/or rubbing the composition onto the skin), the brownish tint, for example, remains visible. The tint can remain visible even after the composition dries on the skin and until it is washed off or otherwise physically removed. An unexpected advantage of this is that it can provide the skin with an immediate tanning appearance (e.g., within about 5, 10, 15, 30 or thirty second, or within 1, 2, 3, 4, 5, 10, 15, 20, or 30 minutes after application to skin or after the composition dries on the skin) before the DHA tanning takes effect. In more particular embodiments, the color additive includes an organic additive, an inorganic additive, a mica, or any combination thereof. For instance, a preferred non-limiting embodiment includes a mixture of at least one or more (e.g. 1, 2, 3, 4, 5, 6, 7, 8, 9, 10 . . . etc.) water-soluble dye(s) and at least one or more (e.g. 1, 2, 3, 4, 5, 6, 7, 8, 9, 10 . . . etc.) pigment(s). In certain aspects, the pigment is water insoluble and/or oil insoluble.

In another embodiment of the present invention, there is disclosed a method of tanning the skin comprising applying the water-in-silicon sunless skin tanning emulsions described above and throughout this specification to skin. The emulsion can be formulated to be applied 1, 2, 3, 4, 5, 6, 7, or more times a day. As noted above, the emulsion can be formulated into a cosmetic composition or product (e.g., sunless skin tanning product).

It is contemplated that any embodiment discussed in this specification can be implemented with respect to any method or composition of the invention, and vice versa. Furthermore, compositions of the invention can be used to achieve methods of the invention.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The terms “about” and “approximately” are defined as being close to as understood by one of ordinary skill in the art, and in one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

The use of sunless tanning compositions have gained more and more popularity over the years. An advantage of sunless tanning compositions includes the possibility of obtaining a tan or darkening the skin without having to expose skin to the damaging sun or artificial UV rays, for example. One of the more widely used tanning agents in sunless tanning products is DHA.

The inventor has discovered compositions that can be used to tan or darken skin that offer advantages over previous compositions. For example, the present compositions and methods allow a user to obtain a homogenous or uniform tan with a composition that can have an improved tactile feeling and visual appearance when compared to previous compositions. In one non-limiting aspect, for instance, the compositions of the present invention can be visually transparent or clear and provide a sensuous, smooth, or silky feel upon application to the skin. The compositions of the present invention can include, for example, a skin tanning agent and a silicone containing surfactant. The composition can also be formulated into a water-in-silicone emulsion. In certain aspects, the composition can also provide the skin with an immediate tanning appearance before the DHA tanning takes effect.

These and other aspects of the present invention are described in further detail in the following sections.

A. Skin Tanning Agents

In a non-limiting aspect of the present invention, there are provided compositions including a sunless skin tanning agent that can be used to tan or darken skin. For purposes of the present specification, the phrases “sunless tanning agent,” “sunless tanner, “skin tanner, “skin tanning agent, “self tanning agent,” “sunless tanning active,” or “self tanning active” can be used interchangeably. Sunless tanning agents can include any compound or material which can stain skin or cause skin pigmentation to darken by exposure to the sunless tanning agent, without the need to expose the skin to sun or artificial ultraviolet light rays.

Non-limiting examples of sunless tanning agents that can be used with the present invention include bronzers (e.g., creams, liquids, sprays, and powders that a person can apply to skin to make the skin look tan, brown, or darker from being in the sun), pigmentation agents (e.g., methoxsalen, trioxsalen, and melanin), dyes, botanical extracts (e.g., silver birch (Betulla alba), and Mahakanni STLC (Eclipta alba)), and chemical compounds (e.g. dihydroxyacetone, erythrulose, lawsone, tyrosine, orjugulone, alpha-hydroxy aldehydes and ketones, glyceraldehyde and related alcohol aldehydes, various indoles, imidazoles, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde). Other sunless tanning agents, including those known and unknown to a person of skill in the art, are also contemplated as being useful with the present invention. Non-limiting examples of the sunless tanning agents are also described in U.S. Pat. Nos. 6,482,397; 6,262,541; 5,559,146; 6,447,760; and 6,443,164.

In certain non-limiting embodiments, dihydroxyacetone (“DHA”) is a preferred sunless tanning agent. DHA colorless sugar (triose carbohydrate) having the chemical formula C₃H₆O₃. It can be prepared by the mild oxidation of glycerol, for example with hydrogen peroxide and a ferrous salt as a catalyst. The chemical formula for DHA is illustrated below:

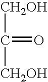

When applied, DHA interacts with dead skin cells located in the stratum corneum of the epidermis. This interaction causes a color change thereby providing a darkening or tanning effect which typically occurs about 2 to 4 hours after application. The color change typically lasts from about five to seven days from the initial application.

In certain aspects, the sunless tanning agents are present in compositions at concentrations at “effective amounts.” Such effective amounts include an amount of the sunless tanning agent which, when applied to skin, causes the skin to darken or tan without the need to expose the skin to natural sunlight or artificial UV light sources, for example. A person of ordinary skill in the art is capable of determining an appropriate amount of the sunless tanning agent in a given composition by using known means (e.g., applying different amounts of particular agents to skin in a testing environment).

B. Silicon Containing Surfactants

The term surfactant is derived from the phrase surface active agent. Surfactants are typically amphiphilic molecules that can be absorbed at various interfaces and can change the properties of the interfaces. Surfactants have wide range applications from forming or stabilizing emulsions, oil recovery, cleansing applications, to efficient delivery of drugs at a desired site in the body.

In one aspect of the present invention, there is disclosed a sunless skin tanning composition that is formulated as a water-in-silicon emulsion. A silicon containing surfactant can be used to facilitate the formation of the emulsion. In a non-limiting aspect, the silicon containing surfactant can include, for example, the following structure:

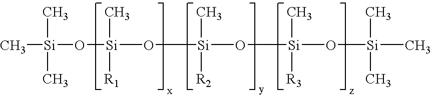

wherein, R₁, R₂, R₃, are independently CH₃, [CH₂]₃—[OCH₂CH₂]_{w}—OH, or [CH₂]ₘ-A, and wherein w is an integer between 2-20, A is: 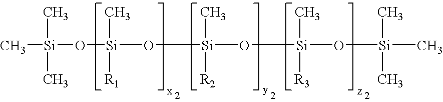
wherein x, y, z, x₂, y₂, z₂, and m are independently an integer between 1 and 1000, 10 and 500, 20 and 100, or 30 and 50, or any integer derivable therein (e.g., see the integers listed from 2-999 above).

In particular aspects of the present invention, the silicon containing surfactant is a polyethylene glycol 12 (PEG-12) dimethicone cross polymer having the following structure: 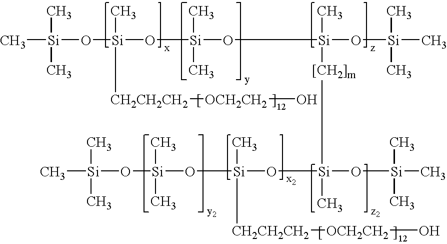
wherein x, y, z, x₂, y₂, z₂, and m are independently an integer between 1 and 1000, 10 and 500, 20 and 100, or 30 and 50, or any integer derivable therein (e.g., see the integers listed from 2-999 above).

The compositions of the present invention can also include a cyclic or non cyclic siloxane compound to aid in the formation of an emulsion. In certain non-limiting aspects, for example, the cyclic siloxane can be cyclomethicone (CAS number 69430-24-6), cyclopentasiloxane (CAS number 6166-86-5), or decamethylcyclopentasiloxane (CAS number 541-02-6). The chemical structures of these compounds are: 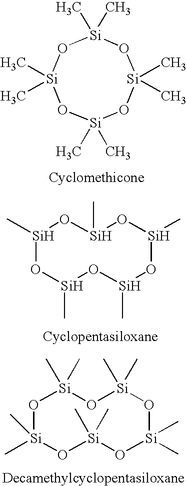
These compounds have excellent spreading, easy rub-out, and lubrication properties. They can also can impart a soft or silky feel to the skin.

A mixture or blend of polyethylene glycol 12 (PEG-12) dimethicone cross polymer and cyclopentasiloxane can be obtained from Dow Corning Corporation under the product name Dow Corning® 9011 Silicone Elastomer Blend (INCI name: Cyclopentasiloxane and PEG-12 Dimethicone Cross polymer). Dow Corning describes this product as a silicone elastomer emulsifier that can be used to prepare water-in-silicone emulsions or multiple emulsions (e.g., water-in-decamethylcyclopentasiloxane in water or water-in-silicon in water). It is a 13% dispersion of high molecular weight silicone elastomer substituted with polyether in Decamethylcyclopentasiloxane. Dow Corning provides the physical and chemical properties of this product as being in liquid form, having a tan color, a specific gravity at 25° C. of 0.96, a viscosity of 100 cP, and a boiling point of >100° C. Formulation flexibility can be achieved by submitting a variety of ingredients in either the aqueous phase or the oil phase, by varying the ratio of the aqueous phase to the oil phase, by increasing or decreasing the Dow Corning 9011® Silicone Elastomer Blend level, or by varying the type or amount of shear that is applied during or after the formation of the emulsion system. The above information concerning this product can be obtained through Dow Corning Corp. (see www.dowcorning.com). The following are formulas for decamethylcyclopentasiloxane and PEG-12 dimethicone cross polymer: 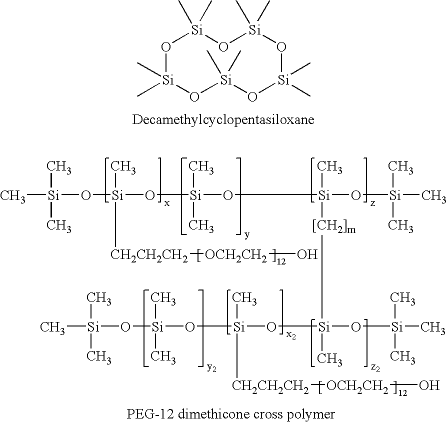
wherein x, y, z, x₂, y₂, z₂, and m are independently an integer between 1 and 1000, 10 and 500, 20 and 100, or 30 and 50, or any integer derivable therein (e.g., see the integers listed from 2-999 above).

Other silicone containing surfactants are contemplated as being useful with the present invention. These surfactants include, for example, poly dimethyl siloxanes that have been modified to include polyether side or pendant chains such as polyethylene oxide chains, polypropylene oxide chains, mixtures of these chains, and polyether chains containing moieties derived from both ethylene oxide and propylene oxide. Additional non-limiting examples include poly dimethyl siloxanes that can be modified to include polyglycerine side chains, alkyl-modified dimethicone copolyols, (i.e., compounds that contain C2-C30 pendant side chains), and dimethicone copolyols include materials having various cationic, anionic, amphoteric, and zwitterionic pendant moieties, and any other silicone containing surfactants that are known to those of ordinary skill in the art.

The inventor also contemplates the use of non-silicon containing surfactants that can be used with the present invention. Non-limiting examples include cationic, anionic, zwitterionic, or nonionic surfactants, or mixtures thereof (Rosen 1988; Rieger 1999). U.S. Pat. No. 6,495,126, for example, provides a non-limiting list of the different types of surfactants that can be used with the present invention. Suitable cationic surfactants include, but are not limited to, DMDAO or other amine oxides, long-chain primary amines, diamines and polyamines and their salts, quaternary ammonium salts, polyoxyethylenated long-chain amines, and quaternized polyoxyethylenated long-chain amines. Non-limiting examples of anionic surfactants include SDS, salts of carboxylic acids (i.e. soaps), salts of sulfonic acids, salts of sulfuric acid, phosphoric and polyphosphoric acid esters, alkylphosphates, monoalkyl phosphate (MAP), and salts of perfluorocarboxylic acids. Examples of zwitterionic surfactants include, but are not limited to, cocoamidopropyl hydroxysultaine (CAPHS) and others which are pH-sensitive and require special care in designing the appropriate pH of the formula (i.e. alkylaminopropionic acids, imidazoline carboxylates, and betaines) or those which are not pH-sensitive (i.e. sulfobetaines, sultaines). Suitable nonionic surfactants can include, but are not limited to, alkylphenol ethoxylates, alcohol ethoxylates, polyoxyethylenated polyoxypropylene glycols, polyoxyethylenated mercaptans, long-chain carboxylic acid esters, alkonolamides, tertiary acetylenic glycols, polyoxyethylenated silicones, N-alkylpyrrolidones, and alkylpolyglycosidases.

In other embodiments, any combination of the surfactants discussed in this document or known to a person of skill in the art is also acceptable. For example, the use of silicone and non-silicone containing surfactants can be used in the same or separate emulsions.

C. Color Additives

The composition of the present invention can also include a color additive. The color additive can be used to provide the composition with a particular color (e.g., white, off-white, light brown, dark brown, tan, creme, caramel, yellow, orange, blue, green, red, etc.). As explained above, and advantage of this is that it can provide the skin with an immediate tanning appearance before the DHA tanning takes effect.

The color additive can include be an organic additive, an inorganic additive, a mica, or any combination thereof. Non-limiting examples of color additives that can be used in combination with the present invention are described in the International Cosmetic Ingredient Dictionary and Handbook, 10^{th} Edition (2004), which is incorporated into this document by reference. Examples of organic color additives include water soluble dyes (e.g.: FD&C Red 3 or 40; FD&C Yellow 5 or 6; FD&C Blue 1 or 2; FD&C Green 3; D&C Green 5, 6, or 8; D&C Orange 4; D&C Red 4, 6, 17, 22, 28, or 33; D&C Violet 2; D&C Yellow 8, 10, or 11), oil soluble dyes, and pigments (e.g., lakes (e.g.: Blue 1 Lake; Ext. Yellow 7 Lake; Green Lake; Orange 4; 5; or 10 Lake; Red 4, 6, 7, 21, 22, 27, 28, 30, 31, 33, 34, 36, or 40 Lake; and Yellow 5, 6, 7, or 10 Lake), carmines, Timiron Splendid Copper, and SunShine Super Gold).

Inorganic additives can produce “earthy” tones which can be used to achieve muted colors. For instance, iron oxides (e.g., red, black, and yellow oxides) in combination with titanium dioxide can be used to achieve a brownish tint. Non-limiting examples of other inorganic additives include pigments such manganese, ultramarines, titanium dioxides, ferric ferrocyanide, and chromium oxide.

Micas use light reflection, refraction, and transmission to exhibit their effects. Micas can be made by coating small platelets of mica with selected dyes and/or pigments. An important aspect of micas is their size which can affect its ability to bend and reflect light. For instance, a person of ordinary skill in the art would recognize that smaller sizes can provide a smooth sheen while medium and larger sizes provide a satin and sparkling appearance, respectively.

D. Source of Compounds, Agents, and Active Ingredients

The compounds, agents, and active ingredients that are described in the claims and specification can be obtained by any means known to a person of ordinary skill in the art. In a non-limiting embodiment, for example, the compounds, agents, and active ingredients can be isolated by obtaining the source of such compounds, agents, and active ingredients. In many instances, the compounds, agents, and active ingredients are commercially available. For example, DHA can be obtained through any number of companies including ScienceLab.com, Inc., located in Kingwood, Tex. (also see www.sciencelab.com). Silicone oils and silicone based surfactants can be purchased from a variety of vendor. For example Dow Corning 9011® can be purchased from Dow Corning Corp. (see www.dowcoming.com) or from authorized distributors such as ChemCentral Corp. in Dallas Tex., or Ashland Dist. (Garland) Company Division, in Garland Tex.

Additionally, the compounds, agents, and active ingredients can be purified by any number of techniques known to a person of ordinary skill in the art. Non-limiting examples of purification techniques include Polyacrylamide Gel Electrophoresis, High Performance Liquid Chromatography (HPLC), Gel chromatography or Molecular Sieve Chromatography, and Affinity Chromatography. In other aspects, the compounds, agents, and active ingredients can be obtained by chemical synthesis or by recombinant means by using conventional techniques. See, for example, Stewart and Young, (1984); Tam et al., (1983); Merrifield, (1986); and Barany and Merrifield (1979), Houghten (1985).

E. Modifications and Derivatives

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non limiting examples of the types modifications that can be made to the compounds and structures disclosed throughout this document include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom.

F. Equivalents

Known and unknown equivalents to the specific compounds, agents, and active ingredients discussed throughout this specification can be used with the compositions and methods of the present invention. The equivalents can be used as substitutes for the specific compounds, agents, and active components. The equivalents can also be used to add to the methods and compositions of the present invention. A person of ordinary skill in the art would be able to recognize and identify acceptable known and unknown equivalents to the specific compounds, agents, and active ingredients without undue experimentation.

G. Compositions

A person of ordinary skill would recognize that the compositions of the present invention can include any number of combinations of compounds, agents, and/or active ingredients, or derivatives therein. It is also contemplated that that the concentrations of the compounds, agents, and/or active ingredients can vary. In other non-limiting embodiments, for example, the compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the compounds, agents, active ingredients, or derivatives that are mentioned throughout the specification and claims. In non-limiting aspects, the percentage can be calculated by weight or volume of the total weight or volume of the emulsion or composition. A person of ordinary skill in the art would understand that the concentrations can vary depending on the addition, substitution, and/or subtraction of the compounds, agents, or active ingredients, to the disclosed methods and compositions.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, phenonip, or combinations thereof.

H. Cosmetic Vehicles

The present compositions are effective in all types of cosmetic vehicles. Non-limiting examples of suitable cosmetic vehicles include emulsions, creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

In preferred embodiments, the cosmetic vehicle is an emulsion. Non-limiting examples of emulsions include oil-in-water emulsions, water-in-oil emulsions, and water-in-silicon emulsions. Emulsions and methods of making the same are well known in the art (Sjoblom 1996; Sjoblom 2001; Sjoblom 2002).

I. Cosmetic Products

The composition of the present invention can also be used in many cosmetic products including, but not limited to, sunless skin tanning products, moisturizing creams, skin benefit creams and lotions, softeners, day lotions, gels, ointments, foundations, night creams, lipsticks, cleansers, toners, masks, or other known cosmetic products or applications. Additionally, the cosmetic products can be formulated as leave-on or rinse-off products.

J. Additional Compounds

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, emollients, anti-irritants, vitamins, trace metals, anti-microbial agents, botanical extracts, fragrances, dyes and color ingredients, structuring agents, and/or emulsifiers (see U.S. Pat. No. 6,290,938).

1. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, ascorbic acid, ascorbyl palmitate, aspartic acid, avocado (persea gratissima) oil, barrier sphingolipids, butyl alcohol, beeswax, behenyl alcohol, beta-sitosterol, BHT, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (asminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methylsilanol PCA, microcrystalline wax, mineral oil, mink oil mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

2. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

3. Compounds Having Ultraviolet Light Absorbing Properties

Non-limiting examples of compounds that have ultraviolet light absorbing properties that can be used with the compounds of the present invention include benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

4. Preservatives

Non-limiting examples of preservatives that may used with compositions of the invention include phenonip™, and/or any of its constituents phenoxyethanol, methylparaben, butylparaben, ethylparaben, propylparaben, additionally Suttocide®, Germaben™, LiquiPar potassium sorbate, and/or rosemary oleoresin may be used.

5. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof. Other non-limiting examples can be found in International Cosmetic Ingredient Dictionary, 10th edition, 2004, which is incorporated by reference.

6. Emulsifiers

In certain preferred aspects of the present invention, the compositions do not include an emulsifier. In other aspects, however, the compositions can include one or more emulsifiers. Emulsifiers can reduce the in interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof. Other non-limiting examples can be found in International Cosmetic Ingredient Dictionary, 10th edition, 2004, which is incorporated by reference.

7. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In preferred aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in International Cosmetic Ingredient Dictionary, 10th edition, 2004, which is incorporated by reference as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich. and those described in International Cosmetic Ingredient Dictionary, 10th edition, 2004.

8. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

9. Thickening Agents

Thickening agents, including thickener or gelling agents, include substances which that can increase the viscosity of a composition. Preferred thickeners includes those that can increase the viscosity of a composition without substantially modifying the efficacy of the active ingredient within the composition. Thickeners can also increase the stability of the compositions of the present invention.

Non-limiting examples of thickening agents that can be used in the context of the present invention include hydrogenated polyisobutene or trihydroxystearin or combination of both. Other examples include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Tenth Edition, 2004). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C₃₀ straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

10. Additional Compounds and Agents

Non-limiting examples of additional compounds and agents that can be used with the compositions of the present invention include, vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

K. Kits

In further embodiments of the invention, there is a provided a kit. Any of the compositions, compounds, agents, or active ingredients described in this specification may be comprised in a kit. In a non-limiting example, a kit can include a sunless tanning agent and additional compound or cosmetic product, or both. The sunless tanning agent can be comprised in a composition or sunless tanning product.

The container means of the kits can include a bottle, dispenser, package, compartment, or other container means, into which a component may be placed. Where there is more than one component in the kit (they may be packaged together), the kit also will generally contain a second, third or other additional containers into which the additional components may be separately placed. The kits of the present invention also can include a means for containing the components in close confinement for commercial sale. Such containers may include injection or blow-molded plastic containers into which the desired bottles, dispensers, or packages are retained. For example, a kit of the present invention may include a container that has at least 2, 3, 4, 5, or more separated compartments. One compartment may include a sunless tanning product while the other compartment includes a separate cosmetic product. Alternatively, a kit may include separate containers where one container includes a sunless tanning product while a second container includes a separate cosmetic product.

A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions, for example.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Non-Limiting Examples of Sunless Tanning Compositions

Non-limiting examples of certain embodiments of sunless tanning compositions of the present invention are described in tables 1 and 2. The ingredients in these tables were formulated into a sunless tanning gel that included DHA as the sunless tanning agent and the Dow Corning 9011® mixture as an emulsifier.

TABLE 1*

| Phase | Ingredient | % | g |
|---|---|---|---|
| A | Water | 34.80 | 348.00 |
| | Magnesium Sulfate | 2.00 | 20.00 |
| | Disodium EDTA | 0.20 | 2.00 |
| | Dihydroxyacetone | 5.00 | 50.00 |
| B | Butylene Glycol | 10.0 | 100.00 |
| | Glycerin | 5.0 | 50.00 |
| | Propylene Glycol | 10.0 | 100.00 |
| C | PEG 12 (Carbo Wax) | 3.0 | 30.00 |
| | Phenonip | 1.0 | 10.00 |
| | Cyclomethicone | 15.0 | 150.00 |
| | Dimethicone/cyclomethicone | 7.0 | 70.00 |
| | Dow Corning 9011 | 7.0 | 70.00 |
| | Total | 100.0 | 1000.00 |

*Procedure for making: Mix phase A in a glass beaker. Heat phase A to 28 to 30° C. Mix phase B in a separate glass beaker until all ingredients are homogeneous and clear. Add phase B to phase A and mix until homogeneous. In a separate beaker mix phase C together and add phase A and B to C slowly and mix until uniform. Batch becomes very thick. Switch to a sweep type mix and mix to reduce aeration and mix until batch is uniform.

The table 2 composition includes a mixture of water-soluble dyes and non-water/non-oil soluble pigments that provides the composition with a brownish tint. After the composition is applied to (e.g., spread on and/or rubbed out) and dried on the skin, the brownish tint remains visible. This provides the skin with an immediate tanning appearance before the DHA tanning takes effect. The brownish tint remains visible on the skin until it is washed off or otherwise physically removed.

TABLE 2*

| Phase | Ingredient** | % | g |
|---|---|---|---|
| A | Water | 27.60 | 276.00 |
| | Magnesium Sulfate | 3.00 | 30.00 |
| | Disodium EDTA | 0.20 | 2.00 |
| | Dihydroxyacetone | 7.50 | 75.00 |
| | FD&C Yellow #6 1% c.s. | 3.40 | 34.00 |
| | FD&C Yellow #5 1.5% c.s. | 0.15 | 1.50 |
| | FD&C Blue #1 1.75% c.s. | 0.10 | 1.00 |
| | D&C Red #33 .0.25% c.s. | 0.30 | 3.00 |
| B | Butylene Glycol | 10.0 | 100.00 |
| | Glycerin, 99% | 5.0 | 50.00 |
| | Propylene Glycol | 10.0 | 100.00 |
| | PEG 12 (Carbo Wax) | 3.0 | 30.00 |
| | Liquapar MEP | 1.0 | 10.00 |
| C | Cyclopentasiloxan | 13.00 | 130.00 |
| | Dimethicone/cyclomethicone | 7.0 | 70.00 |
| | Dow Corning 9011 | 8.0 | 80.00 |
| D | Timiron Splendid Copper | 0.125 | 1.250 |
| | SunShine Super Gold | 0.125 | 1.250 |
| | Pure Aura Fragrance | 0.50 | 5.00 |
| | Total | 100.0 | 1000.00 |
| *Procedure for making: Mix phase A in a glass beaker. Heat phase A to 28 to 30° C. Mix phase B except colors in a separate glass beaker until all ingredients are homogeneous and clear. Add colors in phase B and mix. Add phase B to phase A and mix until homogeneous. In a separate beaker mix phase C together and add phase A and B to C slowly and mix until uniform. Batch | | | |
| # becomes very thick. Add phase D mixing with a sweep type mix and mix until the pearls and pigments are uniform within the batch. | | | |

**Ingredients can be obtained from companies in parenthesis: 1. Liquapar MEP (International Specialty Products); 2. Timiron Splendid Copper (EMD Chemicals); 3. Sunshine Super Gold (Sun Chemicals); 4. Pura Aura Fragrance (Fragrance Resources).

### Example 2

### Proposed Testing Procedure

The formulations in tables 1 and 2 include a known sunless skin tanning agent, DHA. A person of ordinary skill in the could set up an appropriate testing procedure to determine the efficacy of these formulations and/or compare the efficacy of these formulations with other sunless skin tanning formulations without undue experimentation.

For example, a study could include using individuals/panelists. At an initial visit, a number of testing sites on an individual's skin can be marked on the volar forearm. The skin color of the testing cites can be measured prior to application of a formulation(s). The color of the skin can be measured at each site with a Minolta Chromameter. This instrument provides 3 values—L*, which is a measure of light or dark; a* which is a measure of red; and b* which is a measure of yellow. Dihydroxyacetone (DHA), which is a known active ingredient in sunless tanning lotions, induces a yellow brown stain of the skin. This color change is observed as a change in the L* and b* values. Because the L* value can also be affected by other influences such as change in blood flow, the b* value is typically evaluated.

The sunless skin tanning formulation can be applied under occlusion patches to each site (e.g., one control and four test sites). The panelists can be instructed to remove the occlusive patches 4 to 6 hours later. Color measurements can then be taken again after 4 to 6 hours later or the following morning (e.g., 24 hours after application). One site can receive no test material and can be used as an untreated control. The change in brown color (b*) can be determined as a percentage of the baseline b* value.

### REFERENCES

The following references, to the extent that they provide exemplary procedural or other details supplementary to those set forth herein, are specifically incorporated herein by reference.
U.S. Pat. No. 2,798,053
U.S. Pat. No. 3,755,560
U.S. Pat. No. 4,421,769
U.S. Pat. No. 4,509,949
U.S. Pat. No. 4,599,379
U.S. Pat. No. 4,628,078
U.S. Pat. No. 4,835,206
U.S. Pat. No. 4,849,484
U.S. Pat. No. 5,011,681
U.S. Pat. No. 5,087,445
U.S. Pat. No. 5,100,660
U.S. Pat. No. 5,559,146
U.S. Pat. No. 6,262,541
U.S. Pat. No. 6,290,938
U.S. Pat. No. 6,443,164
U.S. Pat. No. 6,447,760
U.S. Pat. No. 6,482,397
U.S. Pat. No. 6,495,126
Barany and Merrifield, In: The Peptides, Gross and Meienhofer (Eds.), Academic Press, NY, 1-284, 1979.
International Cosmetic Ingredient Dictionary, 10th edition, 2004.
Houghten et al., Infect. Immun., 48(3):735-740., 1985.
McCutcheon's, Detergents and Emulsifiers, North American Edition (1986).
Merrifield, Science, 232(4748):341-347, 1986.
Remington's Pharmaceutical Sciences, 18th Ed. Mack Printing Company, 1289-1329, 1990.
Rieger et al., Br. J. Dermatol., 140(3):497-504, 1999.
Rosen et al., Cell 41:813, 1988.
Sjoblom, Emulsions—a Fundamental and Practical Approach, 2002.
Sjoblom, Encyclopedic Handbook of Emulsion Technology, 2001.
Sjoblom, In: Emulsions and Emulsion Stability, 1996.
Stewart and Young, In: Solid Phase Peptide Synthesis, 2d. ed., Pierce Chemical Co., 1984.
Tam et al., J. Am. Chem. Soc., 105:6442, 1983.

What is claimed is:

1. A water-in-silicon sunless skin tanning emulsion comprising:
(a) a skin tanning agent and;
(b) a silicon containing compound comprising the formula: 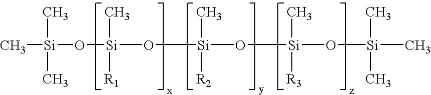
wherein,
R₁, R₂, R₃, are independently CH₃, [CH₂]₃—[OCH₂CH₂]_{w}—OH, or [CH₂]ₘ-A,
wherein,
w is an integer between 2-20;
A is: 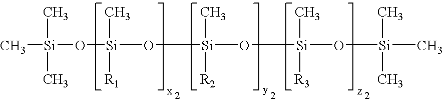
and x, y, z, x₂, y₂, z₂, and m are independently an integer between 1 and 100.

2. The water-in-silicon sunless skin tanning emulsion of claim 1, wherein the silicon containing compound is a dimethicone crosslinked polymer comprising the formula: 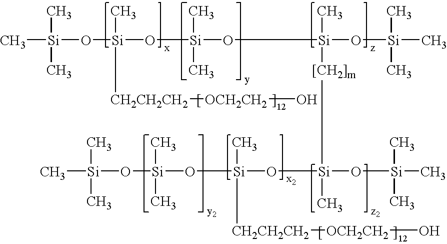
wherein x, y, z, x₂, y₂, z₂, and m are independently an integer between 1 and 100.

3. The water-in-silicon sunless skin tanning emulsion of claim 1, wherein the emulsion comprises from about 2% to about 10% by weight of the silicon containing compound relative to the total weight of the emulsion.

4. The water-in-silicon sunless skin tanning emulsion of claim 1, wherein the skin tanning agent is dihydroxyacetone (DHA).

5. The water-in-silicon sunless skin tanning emulsion of claim 1, wherein the emulsion comprises from about 5% to about 10% by weight of the skin tanning agent relative to the total weight of the emulsion.

6. The water-in-silicon sunless skin tanning emulsion of claim 1, wherein the emulsion is transparent.

7. The water-in-silicon sunless skin tanning emulsion of claim 1, wherein the emulsion is comprised in a cosmetic composition.

8. The water-in-silicon sunless skin tanning emulsion of claim 7, wherein the composition is a gel.

9. The water-in-silicon sunless skin tanning emulsion of claim 1, further comprising a cyclic siloxane.

10. The water-in-silicon sunless skin tanning emulsion of claim 9, wherein the cyclic siloxane is cyclomethicone.

11. The water-in-silicon sunless skin tanning emulsion of claim 9, wherein the cyclic siloxane is cyclopentasiloxane.

12. The water-in-silicon sunless skin tanning emulsion of claim 9, wherein the cyclic siloxane is decamethylcyclopentasiloxane.

13. The water-in-silicon sunless skin tanning emulsion of claim 9, further comprising at least two cyclic siloxanes.

14. The water-in-silicon sunless skin tanning emulsion of claim 13, where the at least two cyclic siloxanes are cyclomethicone and cyclopentasiloxane.

15. The water-in-silicon sunless skin tanning emulsion of claim 13, where the at least two cyclic siloxanes are cyclomethicone and decamethylcyclopentasiloxane.

16. The water-in-silicon sunless skin tanning emulsion of claim 9, wherein the emulsion comprises from about 5% to about 30% by weight of the cyclic siloxane relative to the total weight of the emulsion.

17. The water-in-silicon sunless skin tanning emulsion of claim 1, further comprising a mono or dihydric alcohol.

18. The water-in-silicon sunless skin tanning emulsion of claim 17, wherein the emulsion comprises a monohydric alcohol.

19. The water-in-silicon sunless skin tanning emulsion of claim 18, wherein the monohydric alcohol is ethanol or isopropanol.

20. The water-in-silicon sunless skin tanning emulsion of claim 17, wherein the emulsion comprises a dihydric alcohol.

21. The water-in-silicon sunless skin tanning emulsion of claim 20, wherein the dihydric alcohol is butylene glycol, propylene glycol, or isopentyldiol.

22. The water-in-silicon sunless skin tanning emulsion of claim 17, wherein the emulsion comprises from about 3% to about 12% by weight of the monohydric or dihydric alcohol relative to the total weight of the emulsion.

23. The water-in-silicon sunless skin tanning emulsion of claim 1, further comprising a gellant.

24. The water-in-silicon sunless skin tanning emulsion of claim 22, wherein the gellant is a dibenzylidene alditol, a carboxylated salt, a polysaccharide, a protein/polysaccharide complex, or mixtures thereof.

25. The water-in-silicon sunless skin tanning emulsion of claim 1, further comprising a color additive.

26. The water-in-silicon sunless skin tanning emulsion of claim 25, wherein the color additive imparts a brown color to the emulsion.

27. The water-in-silicon sunless skin tanning emulsion of claim 25, wherein the color additive is a mixture of at least one water-soluble dye and at least one pigment.

28. The water-in-silicon sunless skin tanning emulsion of claim 27, wherein the at least one pigment is water insoluble and oil insoluble.

29. The water-in-silicon sunless skin tanning emulsion of claim 27, wherein the color additive includes about 0.001% to about 0.005% by weight of the at least one water-soluble dye.

30. The water-in-silicon sunless skin tanning emulsion of claim 27, wherein the color additive includes about 0.1% to about 0.5% by weight of the at least one pigment.

31. The water-in-silicon sunless skin tanning emulsion of claim 27, wherein the color additive includes at least two water soluble dyes selected from the group consisting of: FD&C Yellow #5, FD&C Yellow #6, FD&C Blue #1, and D&C Red #33.

32. The water-in-silicon sunless skin tanning emulsion of claim 27, wherein the color additive includes at least two pigments.

33. The water-in-silicon sunless skin tanning emulsion of claim 25, wherein the color additive is formulated to stain the skin after the emulsion is applied to and dried on the skin, and wherein the color additive stain is removable by washing the skin.

34. The water-in-silicon sunless skin tanning emulsion of claim 33, wherein the color additive stains the skin brown.

35. The water-in-silicon sunless skin tanning emulsion of claim 1, wherein the skin tanning agent is formulated to stain the skin brown and wherein the skin tanning agent stain is not removable by washing the skin.

36. The water-in-silicon sunless skin tanning emulsion of claim 35, wherein the skin tanning agent is dihydroxyacetone.

37. The water-in-silicon sunless skin tanning emulsion of claim 26, wherein the brown color of the emulsion is formulated to remain visible after the emulsion is applied to and dried on skin.

38. The water-in-silicon sunless skin tanning emulsion of claim 25, wherein:
(1) the color additive is formulated to stain the skin brown and wherein the color additive stain is removable by washing the skin; and
(2) the skin tanning agent is formulated to stain the skin brown and wherein the skin tanning agent stain is not removable by washing the skin.

39. The water-in-silicon sunless skin tanning emulsion of claim 38, wherein the color additive is formulated to stain the skin brown within about thirty minutes after the emulsion is applied to the skin and wherein the skin tanning agent is formulated to stain the skin brown within about six hours after the emulsion is applied to the skin.

40. The water-in-silicon sunless skin tanning emulsion of claim 25, wherein the emulsion imparts a removable and a non-removable sunless tan to the skin, wherein the removable sunless tan can be removed by washing the skin.

41. A method of tanning skin comprising applying the water-in-silicon sunless skin tanning emulsion of claim 1 to the skin.

42. The method of claim 41, wherein the cosmetic composition is formulated into a gel.

43. The method of claim 41, wherein the product is transparent.

44. The method of claim 41, wherein the emulsion includes a color additive.

45. The method of claim 44, wherein the color additive is a mixture of at least one water-soluble dye and at least one pigment.

46. The method of claim 45, wherein the color additive includes about 0.001% to about 0.005% by weight of the emulsion of the at least one water-soluble dye.

47. The method of claim 45, wherein the color additive includes about 0.1% to about 0.5% by weight of the emulsion of the at least one pigment.

48. The method of claim 45, wherein the color additive includes at least two water soluble dyes selected from the group consisting of: FD&C Yellow #5, FD&C Yellow #6, FD&C Blue #1, and D&C Red #33.

49. The method of claim 45, wherein the color additive includes at least two pigments.

50. The method of claim 44, wherein:
(1) the color additive stains the skin brown and wherein the color additive stain is removable by washing the skin; and
(2) the skin tanning agent stains the skin brown and wherein the skin tanning agent stain is not removable by washing the skin.

51. The method of claim 50, wherein the color additive stains the skin brown within about thirty minutes after application of the emulsion to the skin and wherein the skin tanning agent stains the skin brown within about six hours after application of the emulsion to the skin.
